(19) United States
(12) Patent Application Publication
(10) Pub. No.: US 2015/0369823 A1
(43) Pub. Date: Dec. 24, 2015

(54) METHOD TO IDENTIFY PATIENTS THAT WILL LIKELY RESPOND TO ANTI-TNF THERAPY

(71) Applicants: Brendan F. BOYCE, (US); Lianping XING, (US); Christopher T RITCHLIN, (US); Yan XIU, (US)

(72) Inventors: Brendan F. BOYCE, Pittsford, NY (US); Lianping XING, Webster, NY (US); Christopher T. RITCHLIN, Canandaigua, NY (US); Yan XIU, Rochester, NY (US)

(73) Assignee: UNIVERSITY OF ROCHESTER, Rochester, NY (US)

(21) Appl. No.: 14/761,408

(22) PCT Filed: Jan. 10, 2014
(86) PCT No.: PCT/US14/11106

§ 371 Date: Jul. 16, 2015

Related U.S. Application Data
(60) Provisional application No. 61/753,303, filed on Jan. 16, 2013.

(51) Int. Cl. G01N 33/68

(52) U.S. Cl. CPC G01N 33/6893; G01N 2333/70578; G01N 2800/102

(57) ABSTRACT

The present invention identifies various methods for the identification of patients that suffer from an immune system disorder and are likely to benefit from anti-TNF therapy. Because of the significant cost of anti-TNF therapy and the high rate of ineffectiveness of anti-TNF therapy for treating immune disorders such as rheumatoid and psoriatic arthritis, the present invention will improve the delivery of effective therapies to patients in need of either anti-TNF therapy or alternative therapies.

This application claims the benefit of U.S. Provisional Patent Application Ser. No. 61/753,303, filed Jan. 16, 2013, which is hereby incorporated by reference in its entirety.

This invention was made with government support under grant AR43510 awarded by the National Institutes of Health (NIAMS). The government has certain rights in this invention.

### FIELD OF THE INVENTION

The present invention relates to a method to identify patients that will likely respond to anti-TNF therapy during treatment of various conditions including immune system disorders.

### BACKGROUND OF THE INVENTION

Approximately 1% of the general population in the US (˜310,000) has rheumatoid arthritis (RA), a chronic inflammatory disease that can affect many tissues and is associated with increased morbidity and mortality, but principally involves synovial joints (Aletaha et al., “Rheumatoid Arthritis Classification Criteria,” Arthritis Rheum. 62(10): 2669-2681 (2010)). Psoriatic arthritis (PsA) is an inflammatory joint disease, which occurs in ˜25% of patients with psoriasis, a chronic skin disease that affects ˜7.5 million adults in the United States (Taylor and Helliwell, “Classification Criteria for Psoriatic Arthritis: Development of New Criteria From a Large International Study,” Arth. Rheum. 54: 2665-2673 (2006)). PsA is also associated with increased morbidity and mortality in affected patients. Both RA and PsA patients experience joint destruction via similar mechanisms. In particular, the synovial inflammation and damage in RA and PsA is driven by excessively high levels of tumor necrosis factor (TNF) in both the circulation and affected joints (Aletaha et al., “Rheumatoid Arthritis Classification Criteria,” Arthritis Rheum. 62(10): 2669-2681(2010); Taylor and Helliwell, “Classification Criteria for Psoriatic Arthritis: Development of New Criteria From a Large International Study,” Arth. Rheum. 54: 2665-2673 (2006)). Anti-TNF treatments have significantly reduced the morbidity and mortality in RA and PsA patients, but only 50-60% of patients respond to anti-TNF treatments and achieve true remission, which is maintained almost exclusively in patients on continuous therapy and is observed in less that 25% of patients (Aletaha et al., “Rheumatoid Arthritis Classification Criteria,” Arthritis Rheum. 62(10): 2669-2681 (2010)). Moreover, about 30% of patients respond to methotrexate, an oral disease-modifying agent, and typically they do not require additional therapies.

A critical gap in the treatment approach for these two forms of arthritis is the inability to predict which patients will respond to anti-TNF agents prior to initiation of therapy (Geiler et al., “Anti-TNF Treatment in Rheumatoid Arthritis,” Curr. Pharm. Des. 17(29): 3141-3154 (2011)). At present, patients get started on anti-TNF treatment if they do not respond to methotrexate and hydroxychloroquine treatment, which are typically given along with corticosteroids early on in the course of the disease. Several attempts have been made to develop biomarkers that will help identify patients with forms of RA that will respond to currently available anti-TNF therapies, but all of these have been unsuccessful to date. The lack of a predictive marker is of significant importance because patients are required to be on anti-TNF treatment for a minimum of 4 months before they can be changed to an alternative treatment and these agents are very costly ($16-20,000 per year). Thus, a significant fraction of patients will experience persistent joint pain and attendant joint destruction. A diagnostic test that can identify those RA and PsA patients who are likely to respond to anti-TNF treatment, and conversely identify those who are more likely to benefit from treatment with a non-anti-TNF agent, is a major unmet need.

The present invention is directed to overcoming these and other deficiencies in the art.

### SUMMARY OF THE INVENTION

A first aspect of the present invention relates to a method for identifying an individual that has an immune disorder and is likely to respond to anti-TNFα therapy that involves obtaining a biological sample from the individual and determining a level of TNF Receptor-Associated Factor 3 (“TRAF3”) in the sample, wherein an elevated TRAF3 level identifies the individual as likely to respond to anti-TNFα therapy.

A second aspect of the present invention relates to a method of identifying an individual that has an immune disorder and is likely to respond to anti-TNFα therapy that involves obtaining a biological sample from the individual, labeling TRAF3 in the sample with a TRAF3 binding reagent, and determining a level of TRAF3 in the sample based on said labeling, wherein an elevated TRAF3 level identifies the individual as likely to respond to anti-TNFα therapy.

A third aspect of the present invention relates to a method of treating a patient for an immune disorder that involves determining a level of TRAF3 in a patient sample, and administering to the patient a suitable therapeutic regimen to treat the immune disorder, wherein the suitable therapeutic regimen is selected based on the determined TRAF3 level in the patient sample.

The accompanying Examples demonstrate that a substantial fraction (˜60%) of arthritic patients, both rheumatoid and psoriatic arthritis patients, exhibit elevated TRAF3 levels compared to healthy individuals (normal or control TRAF3 levels) and a second subset of these arthritic patients who also exhibit normal TRAF3 levels. Moreover, for patients that were successfully treated with anti-TNF therapy, their TRAF3 levels were at levels that are lower than the control TRAF3 levels in healthy individuals. These data provide convincing evidence that TRAF3 represents a predictive biomarker to identify those patients that will respond to anti-TNF therapy.

### BRIEF DESCRIPTION OF THE DRAWINGS

FIG. 1 shows TRAF3 fluorescence intensities in different subsets of monocyte from healthy controls. Human mononuclear cells purified from peripheral blood from 3 healthy subjects were stained with PE-CD16 and APC-CD14 mAbs. The cells then were fixed and permeabilized with BD Cytofix/Cytoperm buffers followed by TRAF3 Ab staining TRAF3 intensities in CD16+CD14−, CD16−CD14+, and CD16+CD14+ monocyte subsets were analyzed by BD LSRII and Flowjo. Background staining is shown as grey. No differences are observed in TRAF3 fluorescence intensity among the 3 populations of monocytes.

FIG. 2 shows TRAF3 fluorescence intensities in RA patients and healthy controls. Mononuclear cells purified from blood samples from 5 healthy controls and 10 RA patients were stained with PE-CD16 and APC-CD14 mAbs. The cells then were fixed and permeabilized with BD Cytofix/Cytoperm buffers followed by TRAF3 Ab staining TRAF3 intensities in CD16+CD14−, CD16−CD14+, and CD16+CD14+ monocyte subsets were analyzed using BD LSRII and Flowjo.

FIG. 3 shows TRAF3 expression levels in PBMCs are increased in up to ˜60% of RA patients.

FIG. 4 shows TRAF3 expression levels are decreased in PBMCs from RA patients who had a good response to anti-TNF treatment.

FIG. 5 shows TRAF3 expression levels in PBMCs are increased in ˜60% of psoriatic patients with arthritis. PsA patients could be divided into two distinct groups based on TRAF3 intensities on monocyte subsets. Purified human mononuclear cells from ten PsA patient were stained with PE-CD16 and APC-CD14 mAbs, and then the cells were fixed and permeabilized with BD Cytofix/Cytoperm Buffers followed by TRAF3 Ab staining TRAF3 intensities on CD16+CD14−, CD16−CD14+, and CD16+CD14+ monocyte subsets were analyzed by BD LSRII and Flowjo.

### DETAILED DESCRIPTION OF THE INVENTION

One aspect of the present invention relates to a method to identify an individual with an immune disorder with increased likelihood of response to anti-TNFα therapy.

According to one embodiment, the method includes obtaining a biological sample from the individual and determining a level of TRAF3 in the sample, wherein an elevated TRAF3 level identifies the individual as likely to respond to anti-TNFα therapy.

According to a second embodiment, the method includes obtaining a biological sample from the individual, labeling TRAF3 in the sample with a TRAF3 binding reagent, and determining a level of TRAF3 in the sample based on said labeling, wherein an elevated TRAF3 level identifies the individual as likely to respond to anti-TNFα therapy.

As used herein, the term “labeling” is used in reference to a state of TRAF3 that allows the TRAF3 to be assayed. For example, molecules can be modified, or labeled, such that the molecule can be visualized using detection methods well known in the art. Labeling, however, is not required for determining the level of TRAF3 in the sample.

TRAF3 is associated with cytoplasmic and/or endosomic fractions of the cell. TRAF3 regulates pathways leading to the activation of NFκB and MAP kinases, and plays a central role in the regulation of B-cell survival. TRAF3 inhibits activation of NFκB in response to lymphotoxin-beta receptor stimulation. TRAF3 has also been shown to inhibit TRAF2-mediated activation of NFκB, as well as inhibit non-canonical activation of NFκB via downregulation of NFκB2 proteolytic processing. TRAF3 also plays a role in T-cell dependent immune responses. TRAF3 is a part of signaling pathways leading to the production of cytokines and interferon, and it also is an essential constituent of several E3 ubiquitin-protein ligase complexes.

In accordance with all aspects of the present invention, the terms “individual” and “patient” are used interchangeably and encompass any animal, preferably, a mammal. Exemplary mammalian subjects include, without limitation, humans, non-human primates, dogs, cats, rodents (e.g., mouse, rat, guinea pig), horses, cattle and cows, sheep, and pigs. In preferred embodiments of the present invention the individual is a human.

In accordance with the above aspects of the present invention, an individual having an immune system disorder which may benefit from anti-TNFα therapy is selected prior to obtaining said biological sample. TNF-related pathologies of the immune system include, but are not limited to, acute and chronic immune and autoimmune diseases, such as systemic lupus erythematosus, rheumatoid arthritis, psoriatic arthritis, ankylosing spondylitis, thyroiditis, graft versus host disease, scleroderma, diabetes mellitus, Graves' disease, Beschet's disease and postmenopausal osteoporosis; and inflammatory diseases, such as chronic inflammatory pathologies and vascular inflammatory pathologies, including atherosclerosis, sarcoidosis, inflammatory bowel disease, ulcerative colitis, Crohn's disease, disseminated intravascular coagulation, giant cell arteritis and Kawasaki's pathology.

In certain embodiments, the immune system disorder is selected from the group of inflammatory bowel diseases, rheumatoid arthritis, psoriatic arthritis, and ankylosing spondylitis.

In one embodiment, an individual is selected for screening in accordance with the present invention prior to said individual receiving an anti-TNFα therapy and prior to obtaining said biological sample. The selection of the individual can be based upon prior diagnosis or suspicion that the individual has an immune system disorder of the type described above. Thus, diagnosis can be confirmed separately before screening in accordance with the present invention or, alternatively, diagnosis and screening in accordance with the present invention can be carried out in parallel. In the latter approach, screening for TRAF3 levels can be used as part of the diagnosis.

In another embodiment, an individual is selected for screening in accordance with the present invention after showing an inadequate response to a non-TNFα inhibiting therapeutic agent after at least 4 months of therapy. A non-TNFα inhibiting therapeutic agent may include a disease modifying anti-rheumatic drug (DMARD) such as methotrexate, chloroquine, hydroxychloroquine, sulfasalazine, minocycline, azathioprine, cyclosporine, cyclophosphamide, anakinra, abatacept, rituximab, or tocilizumab.

In another embodiment, an individual is selected for screening in accordance with the present invention after exhibiting one or more clinical symptoms of arthritis or ankylosing spondylitis. In general, symptoms of inflammatory arthritis and ankylosing spondylitis include pain, stiffness and inflammation. In accordance with this aspect of the present invention, the clinical symptoms comprise synovitis in at least one joint, seropositivity for rheumatoid factor, seropositivity for anti-citrullinated protein antibody, abnormal C-reactive protein, abnormal erythrocyte sedimentation rate, current psoriasis or personal or family history of psoriasis, psoriatic nail dystrophy, negative rheumatoid factor, dactylitis, low back pain and stiffness, pain and stiffness in the thoracic region, limited motion in the lumbar spine, limited chest expansion, and history or evidence of iritis or uveitis.

Methods of isolation of biological samples from an individual are well known in the art. The biological sample can be sputum, blood, a blood fraction, tissue or fine needle biopsy sample, urine, stool, peritoneal fluid, or pleural fluid. Preferably, the biological sample is blood or a blood fraction. In one embodiment, the blood fraction comprises peripheral blood mononuclear cells (PBMCs).

Methods for isolation of PBMCs are well known in the art. Typically, blood is collected from subjects into heparinized blood collection tubes by personnel trained in phlebotomy using sterile technique. The collected blood samples can be divided into aliquots and centrifuged, and the thin layer of cells between the erythrocyte layer and the plasma layer, which contains the PBMCs, is removed and used for analysis. Subsets of PBMCs can be purified using techniques including fluorescence activated cell sorting (FACS) and flow cytometry. These methods are well known in the art and include the ability to identify and mechanically sort cells based on whether the cell is labeled with a fluorescent label either directly or indirectly. PBMCs express a number of different markers that allow different subsets of the cells to be identified. In one embodiment, the PBMCs are a CD14+/CD16−, CD14−/CD16+, or CD14+/CD16+ subset of the isolated PBMCs. In certain embodiments, two or all three of these subsets can be analyzed. In still further embodiments, alternative cell surface markers can be used to identify additional subsets of isolated PBMCs for detection of TRAF3 levels.

In a further embodiment, the biological sample is contacted with one or more reagents capable of binding TRAF3 in the sample prior to determining the level of TRAF3 in said sample. In a preferred embodiment, at least one of the one or more reagents is coupled to a detectable label. In general, any suitable detectable label can be utilized. Detectable labels include, without limitation, a fluorescent label, a phosphorescent label, a radioisotope, and the like. Examples of well known labels include, but are not limited to, Fluorescein Isothiocyanate (FITC), Cascade Yellow, Cascade Blue, Phycoerythrin (PE), PE-Texas Red, Allophycocyanin (APC), Cy-5-PE, Cy-7-APC, Peridinin Chlorophyll Protein (PerCP), Biotin, Alkaline Phosphatase, Horseradish Peroxidase, and directly conjugated dyes.

A detectable label can directly or indirectly specifically bind to TRAF3.

In one embodiment, the primary binding reagent is coupled to a detectable label by a secondary binding reagent having binding specificity for the primary binding reagent. A wide range of anti-human, (e.g., anti α, μ, and γ), anti-rat, anti-mouse, anti-guinea pig and anti-rabbit antibodies may be used as secondary binding reagents, for example.

In one embodiment, the one or more reagents comprise a primary TRAF3 binding reagent. Preferably, the primary TRAF3 binding reagent comprises an anti-TRAF3 antibody, binding fragment thereof, or polypeptide or non-polypeptide antibody mimic.

As used herein, the term “antibody” is meant to include intact immunoglobulins derived from natural sources or from recombinant sources, as well as immunoreactive portions (i.e., antigen binding portions) of intact immunoglobulins. The antibodies of the present invention may exist in a variety of forms including, for example, polyclonal antibodies, monoclonal antibodies, intracellular antibodies, antibody fragments (e.g., Fv, Fab, F(ab)₂, diabodies, triabodies, minibodies, etc.), as well as single chain antibodies (scFv), chimeric antibodies and humanized antibodies (Ed Harlow and David Lane, USING ANTIBODIES: A LABORATORY MANUAL (Cold Spring Harbor Laboratory Press, 1999); Huston et al., “Protein Engineering of Antibody Binding Sites: Recovery of Specific Activity in an Anti-Digoxin Single-Chain Fv Analogue Produced in Escherichia coli,” Proc. Nat'l Acad. Sci. USA 85:5879-5883 (1988); Bird et al, “Single-Chain Antigen-Binding Proteins,” Science 242:423-426 (1988), which are hereby incorporated by reference in their entirety).

The present invention also encompasses the use of bispecific humanized antibodies or bispecific antigen-binding fragments (e.g., F(ab′)₂) which have specificity for TRAF3. Techniques for making bispecific antibodies are known in the art (Brennan et al., “Preparation of Bispecific Antibodies by Chemical Recombination of Monoclonal Immunoglobulin G1 Fragments,” Science 229:81-3 (1985); Suresh et al, “Bispecific Monoclonal Antibodies From Hybrid Hybridomas,” Methods in Enzymol. 121:210-28 (1986); Traunecker et al., “Bispecific Single Chain Molecules (Janusins) Target Cytotoxic Lymphocytes on HIV Infected Cells,” EMBO J. 10:3655-3659 (1991); Shalaby et al., “Development of Humanized Bispecific Antibodies Reactive with Cytotoxic Lymphocytes and Tumor Cells Overexpressing the HER2 Protooncogene,” J. Exp. Med. 175:217-225 (1992); Kostelny et al., “Formation of a Bispecific Antibody by the Use of Leucine Zippers,” J. Immunol. 148: 1547-1553 (1992); Gruber et al., “Efficient Tumor Cell Lysis Mediated by a Bispecific Single Chain Antibody Expressed in Escherichia coli,” J. Immunol. 152:5368-74 (1994); and U.S. Pat. No. 5,731,168 to Carter et al., which are hereby incorporated by reference in their entirety).

As used herein, the term “antibody mimic” is intended to refer to molecules capable of mimicking an antibody's ability to bind an antigen, but which are not limited to native antibody structures. Examples of such antibody mimics include, but are not limited to, Adnectins (i.e., fibronectin based binding molecules), Affibodies, DARPins, Anticalins, Avimers, Versabodies, and Aptamers all of which employ binding structures that, while they mimic traditional antibody binding, are generated from and function via distinct molecular structures.

Determining the level of TRAF3 in the sample can be performed by several methods well known in the art. In one embodiment, the level of TRAF3 is determined by measuring the expression level of TRAF3 in the sample based on said contacting.

Measurements of the expression level of TRAF3 can be performed on whole cells in the sample, where the cells are fixed/permeabilized to allow for TRAF3 detection with a labeled reagent. This can be performed using, e.g., flow cytometry. Alternatively, cells of interest can be isolated from a sample, and subcellular fractions can be recovered from the isolated cells of interest. Endosomal fractions can be recovered using, e.g., density gradient centrifugation, free-flow electrophoresis, or immunoseparation techniques. Cytosolic fractions can also be obtained using, e.g., the protocol of Lice et al., “A Method to Separate Nuclear, Cytosolic, and Membrane-Associated Signaling Molecules in Cultured Cells,” Science Signaling 4(203):pI2 (2011), which is hereby incorporated by reference in its entirety.

As described herein, detecting the “expression level” of TRAF3 can be achieved by measuring any suitable value that is representative of the gene expression level. The measurement of gene expression levels can be direct or indirect. A direct measurement involves measuring the level or quantity of RNA or protein. An indirect measurement may involve measuring the level or quantity of cDNA, amplified RNA, DNA, or protein; the activity level of RNA or protein; or the level or activity of other molecules (e.g., a metabolite) that are indicative of the foregoing. The measurement of expression can be a measurement of the absolute quantity of a gene product. The measurement can also be a value representative of the absolute quantity, a normalized value (e.g., a quantity of gene product normalized against the quantity of a reference gene product), an averaged value (e.g., average quantity obtained at different time points or from different samples from a subject, or average quantity obtained using different probes, etc.), or a combination thereof.

When it is desirable to measure the expression level of TRAF3 by measuring the level of protein expression, any protein hybridization or immunodetection based assay known in the art can be used. In a protein hybridization-based assay, an antibody or other agent that selectively binds to a protein is used to detect the amount of that protein expressed in a sample. For example, the level of expression of a protein can be measured using methods that include, but are not limited to, western blot, immunoprecipitation, enzyme-linked immunosorbent assay (ELISA), radioimmunoassay (RIA), fluorescent activated cell sorting (FACS), immunohistochemistry, immunocytochemistry, or any combination thereof. Also, antibodies, aptamers, or other ligands that specifically bind to a protein can be affixed to so-called “protein chips” (protein microarrays) and used to measure the level of expression of a protein in a sample. Alternatively, assessing the level of protein expression can involve analyzing one or more proteins by two-dimensional gel electrophoresis, mass spectroscopy (MS), matrix-assisted laser desorption/ionization-time of flight-MS (MALDI-TOF), surface-enhanced laser desorption ionization-time of flight (SELDI-TOF), high performance liquid chromatography (HPLC), fast protein liquid chromatography (FPLC), multidimensional liquid chromatography (LC) followed by tandem mass spectrometry (MS/MS), protein chip expression analysis, gene chip expression analysis, and laser densitometry, or any combinations of these techniques.

Measuring gene expression by quantifying mRNA expression can be achieved using any commonly used method known in the art including northern blotting and in situ hybridization (Parker et al., “mRNA: Detection by in Situ and Northern Hybridization,” Methods in Molecular Biology 106:247-283 (1999), which is hereby incorporated by reference in its entirety); RNAse protection assay (Hod et al., “A Simplified Ribonuclease Protection Assay,” Biotechniques 13:852-854 (1992), which is hereby incorporated by reference in its entirety); reverse transcription polymerase chain reaction (RT-PCR) (Weis et al., “Detection of Rare mRNAs via Quantitative RT-PCR,” Trends in Genetics 8:263-264 (1992), which is hereby incorporated by reference in its entirety); and serial analysis of gene expression (SAGE) (Velculescu et al., “Serial Analysis of Gene Expression,” Science 270:484-487 (1995); and Velculescu et al., “Characterization of the Yeast Transcriptome,” Cell 88:243-51 (1997), which is hereby incorporated by reference in its entirety). Alternatively, antibodies may be employed that recognize specific duplexes, including DNA duplexes, RNA duplexes, and DNA-RNA hybrid duplexes or DNA-protein duplexes.

Messenger RNA expression can be measured using a nucleic acid amplification assay that is a semi-quantitative or quantitative real-time polymerase chain reaction (RT-PCR) assay. Because RNA cannot serve as a template for PCR, the first step in gene expression profiling by RT-PCR is the reverse transcription of the RNA template into cDNA, followed by its exponential amplification in a PCR reaction. The two most commonly used reverse transcriptases are avilo myeloblastosis virus reverse transcriptase (AMV-RT) and Moloney murine leukemia virus reverse transcriptase (MLV-RT), although others are also known and suitable for this purpose. The reverse transcription step is typically primed using specific primers, random hexamers, or oligo-dT primers, depending on the circumstances and the goal of expression profiling. For example, extracted RNA can be reverse-transcribed using a GeneAmp RNA PCR kit (Perkin Elmer, Calif., USA), following the manufacturer's instructions. The derived cDNA can then be used as a template in the subsequent PCR reaction.

Although the PCR step can use a variety of thermostable DNA-dependent DNA polymerases, it typically employs the Taq DNA polymerase, which has a 5′-3′ nuclease activity but lacks a 3′-5′ proofreading endonuclease activity. An exemplary PCR amplification system using Taq polymerase is TaqMan® PCR (Applied Biosystems, Foster City, Calif.). Taqman® PCR typically utilizes the 5′-nuclease activity of Taq or Tth polymerase to hydrolyze a hybridization probe bound to its target amplicon, but any enzyme with equivalent 5′ nuclease activity can be used. Two oligonucleotide primers are used to generate an amplicon typical of a PCR reaction. A third oligonucleotide, or probe, is designed to detect the nucleotide sequence located between the two PCR primers. The probe is non-extendible by Taq DNA polymerase enzyme, and is labeled with a reporter fluorescent dye and a quencher fluorescent dye. Any laser-induced emission from the reporter dye is quenched by the quenching dye when the two dyes are located close together as they are on the probe. During the amplification reaction, the Taq DNA polymerase enzyme cleaves the probe in a template-dependent manner. The resultant probe fragments disassociate in solution, and signal from the released reporter dye is free from the quenching effect of the second fluorophore. One molecule of reporter dye is liberated for each new molecule synthesized, and detection of the unquenched reporter dye provides the basis for quantitative interpretation of the data.

TaqMan® RT-PCR can be performed using commercially available equipment, such as, for example, the ABI PRISM 7700® Sequence Detection System® (Perkin-Elmer-Applied Biosystems, Foster City, Calif., USA), or the Lightcycler (Roche Molecular Biochemicals, Mannheim, Germany).

In addition to the TaqMan primer/probe system, other quantitative methods and reagents for real-time PCR detection that are known in the art (e.g. SYBR green, Molecular Beacons, Scorpion Probes, etc.) are suitable for use in the methods of the present invention.

To minimize errors and the effect of sample-to-sample variation, RT-PCR is usually performed using an internal standard. The ideal internal standard is expressed at a constant level among different tissues. RNAs most frequently used to normalize patterns of gene expression are mRNAs for the housekeeping genes glyceraldehyde-3-phosphate-dehydrogenase (GAPDH) and β-actin.

Real time PCR is compatible both with quantitative competitive PCR, where internal competitor for each target sequence is used for normalization and quantitative comparative PCR using a normalization gene contained within the sample, or a housekeeping gene for RT-PCR. For further details see, e.g., Heid et al., “Real Time Quantitative PCR,” Genome Research 6:986-994 (1996), which is incorporated by reference in its entirety.

In a further embodiment, the expression of TRAF3 in the biological sample is compared to a control level of TRAF3. Preferably, the control level is the average level or normal range of TRAF3 expression in healthy individuals. As used herein, the term “healthy individual” means the sample is taken from a source (e.g. subject, control subject, cell line) that does not have the condition or disorder being assayed and therefore may be used to determine the baseline for the condition or disorder being measured.

In accordance with these aspects of the present invention, when the level of TRAF3 in the biological sample is determined to be elevated, the individual is then identified as likely to respond to an anti-TNFα therapy. An “anti-TNFα therapy” or “TNFα Inhibitor”, as described herein, is an agent which binds to soluble and/or cell membrane-associated forms of TNFα, and neutralizes, either partially or completely, the proinflammatory effect of TNFα by preventing the binding of TNFα to the TNF-RI/II cell-surface receptors. The TNFα inhibitors can be anti-TNFα antibodies or receptor molecules, but also of other types (e.g., small molecule inhibitors). The essential features of a TNFα inhibitor according to the present invention is the ability to capture TNFα or block TNFα binding to the TNFα receptor on the cells.

Another aspect of the present invention relates to a method of treating a patient for an immune disorder, including the immune disorders described above. This method includes determining a level of TRAF3 in a patient sample and administering to the patient a suitable therapeutic regimen to treat said immune disorder, wherein the suitable therapeutic regimen is selected for administration to the patient based on the TRAF3 level in the patient sample.

The patient sample, as well as methods for determining the level of TRAF3 in the sample, is described above.

In accordance with this aspect of the present invention, when the patient is determined to have an elevated level of TRAF3, the therapeutic regimen includes an anti-TNFα therapeutic agent. In one embodiment, the anti-TNFα therapeutic agent is selected from the group of infliximab, etanercept, adalimumab, certolizumab, or golimumab. The anti-TNFα therapeutic agent can be administered to the patient in accordance with well-known protocols for these therapies. Any known or hereinafter-developed anti-TNFα therapeutic agent can be administered in accordance with the present invention.

The therapeutic regimen may further comprise a non-steroidal anti-inflammatory therapeutic agent or a corticosteroid. Non-steroidal anti-inflammatory agents include, but are not limited to, arylalkanoic acids such as acetaminophen; 2-arylpropionic acids such as ibuprofen, ketorolac and naproxen; n-arylanthranilic acids such as mefenamic acid, meclofenamic acid; oxicams such as piroxicam, meloxicam; arylalkanoic acids such as diclofenac, etodolac, indomethacin, sulindac; and COX-2 inhibitors such as celecoxib. Examples of corticosteroids include dexamethasone, prednisone, prednisolone, 6α-methylprednisolone, fludrocortisone, triamcinolone, paramethasone, betamethasone, and aldosterone. The non-steroidal or steroidal therapeutic agents can also be administered to the patient in accordance with well-known protocols for these therapies.

Also in accordance with this aspect of the present invention, when the patient is determined to have a low level of TRAF3, said therapeutic regimen comprises an anti B-cell, anti T-cell, anti IL-1, anti IL-6, or anti-malarial therapeutic agent. Disease modifying anti-rheumatic drugs (DMARDs) are traditional therapeutics used in treatment regimens of this category. Exemplary therapeutic agents include methotrexate, chloroquine, hydroxychloroquine, sulfasalazine, minocycline, azathioprine, cyclosporine, cyclophosphamide, anakinra, abatacept, rituximab, or tocilizumab. The therapeutic regimen may further comprise a non-steroidal anti-inflammatory therapeutic agent or a corticosteroid as described above. Administration of anti-B cell, anti-T cell, anti-IL1, anti-IL6, antimalarial agents, and/or DMARDs can be administered to the patient in accordance with well known protocols for these therapies.

### EXAMPLES

The following examples are provided to illustrate embodiments of the present invention but they it is no means intended to limit its scope.

### Materials and Methods for Examples 1-2

### Human Mononuclear Cell Purification.

Human peripheral blood mononuclear cells are isolated from whole blood samples by Ficoll gradient (GE 17-1440, Ficoll-Paque PLUS). Briefly, 15 ml of human whole blood are diluted with 15 ml of PBS and carefully layered on 20 ml of Ficoll-Paque PLUS, then centrifuged at 1500 rpm for 30 minutes at 25° C. The interface layer is drawn off using a clean Pasteur pipette, washed with 10× volume of PBS, followed by RBC lysis with ACK buffer (Lonza, 10-548E) and two more washes with PBS.

### Surface Marker Staining.

Purified human mononuclear cells are stained with PE-CD16 (BD Pharmingen™, 555407) and APC-CD14 (BD Pharmingen™, 555399) monoclonal antibodies (mAbs). Purified human mononuclear cells (4×10⁶) are incubated on ice with 100 μl of Ab mix, including 10 μl of PE-CD16 and 10 μl of APC-CD14 mAbs for 30 mins. The cells are then washed with 0.2% BSA/PBS.

### Intracellular TRAF3 Staining.

Cells are fixed and permeabilized with BD Cytofix/Cytoperm Buffer (BD Pharmingen™, 554722) then stained with Rabbit anti-human TRAF3 Ab (ab13721) and visualized using a FITC-anti Rabbit IgG (Santa Cruz, sc-2090). The cells are resuspended with 100 μl of BD Cytofix/Cytoperm Buffer, incubated for 20 min on ice, and washed with 1 ml of 1× BD Perm/Wash Buffer. The cells are resuspended with 100 μl of BD Cytoperm Plus Buffer (BD Pharmingen™, 561651) for 10 min on ice and washed with 1 ml of 1× BD Perm/Wash Buffer. The cells are re-fixed with 100 μl of BD Cytofix/Cytoperm Buffer for 5 min on ice and washed with 1 ml of 1× BD Perm/Wash Buffer. They are then stained with 2 μl of anti-human TRAF3 Ab in 100 μl of 1× BD Perm/Wash Buffer for 30 min on ice, washed with 1 ml of 1× BD Perm/Wash Buffer. They next are stained with 1 μl of FITC-anti Rabbit IgG Ab in 100 μl of 1× BD Perm/Wash Buffer for 30 min on ice, and washed with 1 ml of 1× BD Perm/Wash Buffer. The cells are resuspended in 0.2% BSA/PBS buffer.

### Flow Analysis.

TRAF3 fluorescence intensity in the resuspended cells is assessed using a BD LSRII 12-color cytometry and data are analyzed using Flowjo software.

### Example 1

### Measurement of TRAF3 Levels in Peripheral Blood Monocytes (PBMCs) in Rheumatoid Arthritis Patients

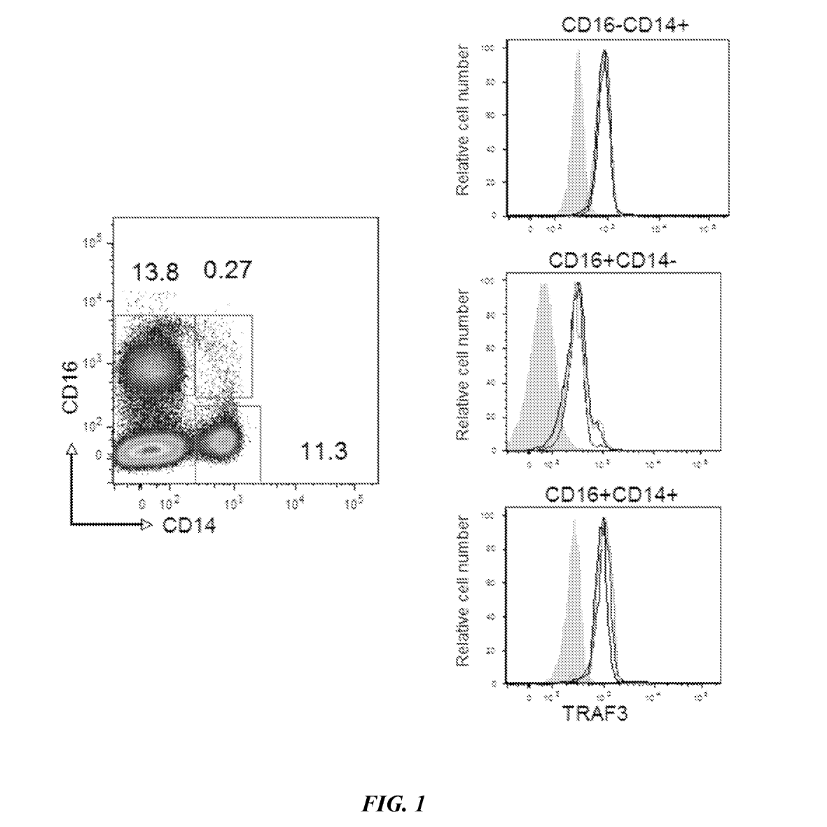

TRAF3 expression levels were first assessed in PBMCs from 3 healthy subjects using FACS. The data showing TRAF3 fluorescence intensities in each sample are illustrated in the right hand panels of FIG. 1 in which each subject's data are illustrated by a blue, green or black line, and 3 populations of PBMCs are depicted as CD16−CD14+, CD16+CD14−, and CD16+CD14+. The left-hand panel shows the percentages of cells staining as CD16−CD14+ (11.3%), CD16+CD14− (13.8%), and CD16+CD14+ (0.27%). These data show that TRAF3 levels are similar in the 3 cell populations in all 3 subjects.

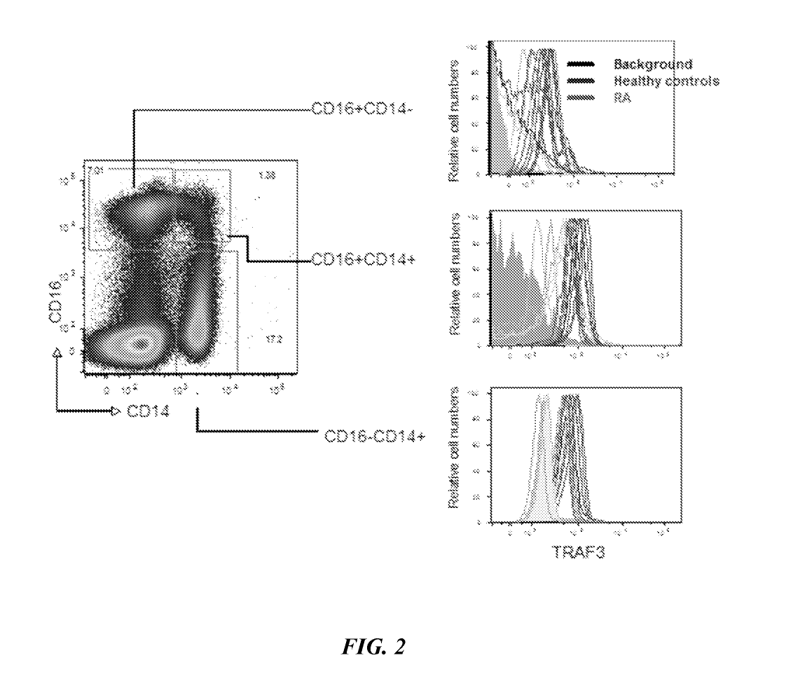

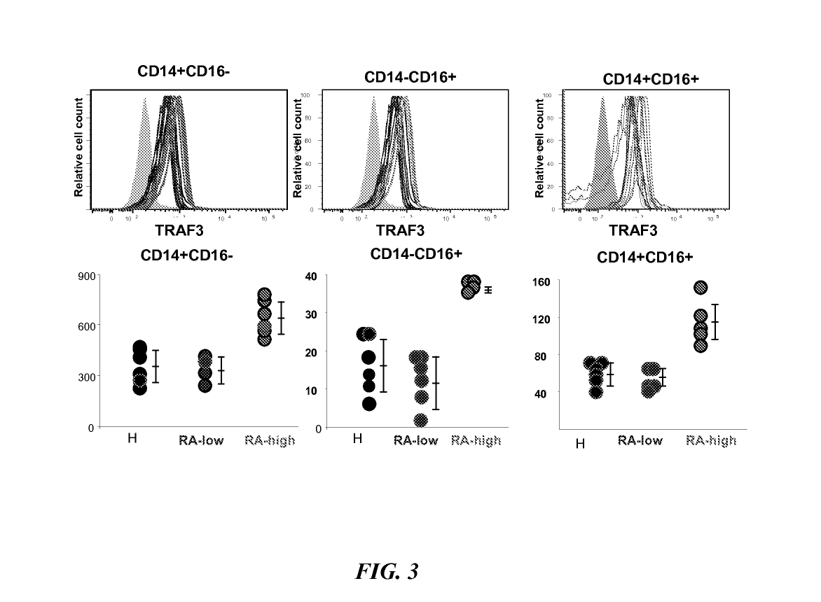

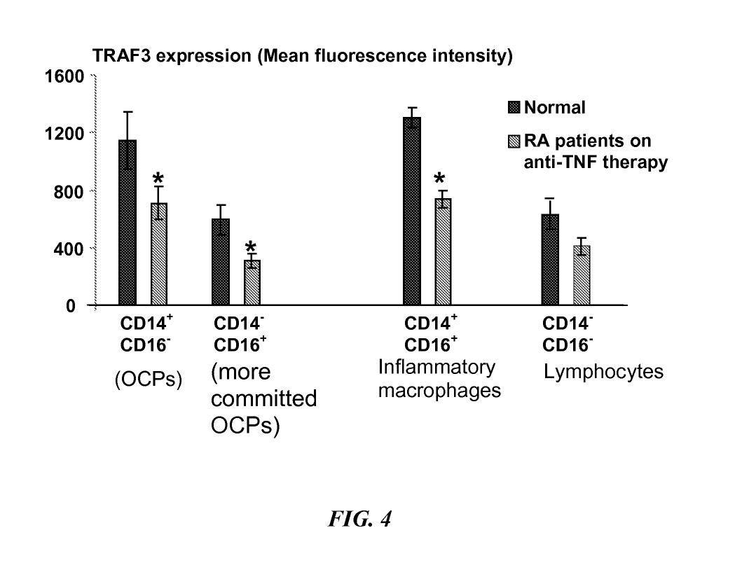

TRAF3 expression levels were then examined in PBMCs from 5 healthy controls and 10 patients with rheumatoid arthritis (RA). The data, illustrated in FIG. 2, show that some RA patients have elevated TRAF3 levels and some have values that overlap with those in the healthy controls. FIG. 3 illustrates these data in a dot plot graph, which shows that TRAF3 levels are increased in up to 60% of PBMCs from these the RA patients. Expression levels of TRAF3 were then assessed in PBMCs from 9 patients who had been treated successfully with anti-TNF therapy to determine if their levels would be low and within the reference range for normal subjects, which based on the present invention would be expected in response to the therapy. FIG. 4 shows that the TRAF3 levels in the PBMCs from these treated RA patients are actually significantly lower than those in normal control subjects.

### Example 2

### Measurement of TRAF3 Levels in Peripheral Blood Monocytes (PBMCs) in Psoriatic Arthritis Patients

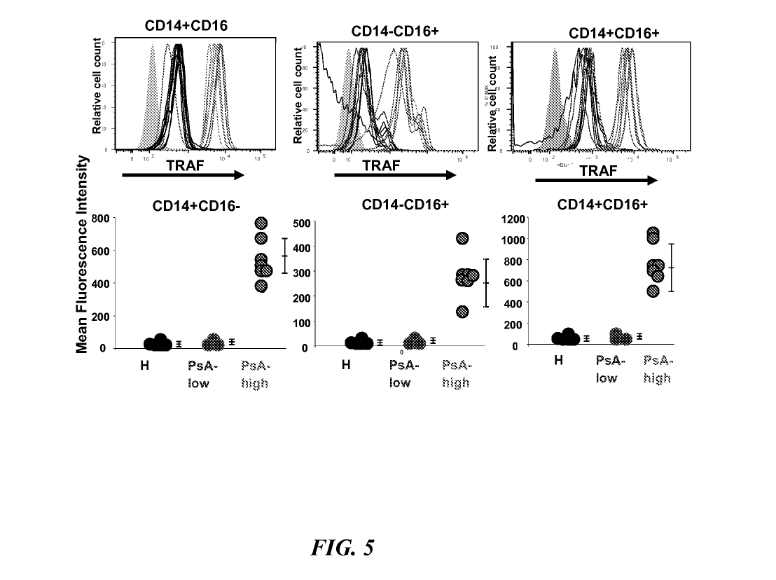

TRAF3 levels were next assessed in 10 patients with psoriatic arthritis (PsA). TRAF3 levels were elevated in the 3 PBMC populations in 6 of the subjects and were low in 4 subjects (FIG. 5), similar to the findings in the RA patients.

From these data, it is believed that TRAF3 levels will be increased in PBMCs from RA and PsA patients in response to increased levels of TNF in their blood and/or bone marrow, that high TRAF3 levels will predict a positive response to anti-TNF therapy, and that TRAF3 levels will decrease to or below the normal range in patients who respond to this treatment. It is also believed that, given these results with RA and PsA patients, the measurement of TRAF3 levels will accurately predict the efficacy of anti-TNFα therapy for patients having various immune disorders of the type described herein.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

What is claimed is:

1. A method for identifying an individual that has an immune disorder and is likely to respond to anti-TNFα therapy, said method comprising:
obtaining a biological sample from the individual;
determining a level of TRAF3 in the sample, wherein an elevated TRAF3 level identifies the individual as likely to respond to anti-TNFα therapy.

2. The method according to claim 1, wherein the immune disorder is selected from the group of inflammatory bowel diseases, rheumatoid arthritis, psoriatic arthritis, and ankylosing spondylitis.

3. The method according to claim 2, wherein the immune disorder is rheumatoid arthritis (RA) or psoriatic arthritis (PsA).

4. The method according to claim 1, wherein the biological sample comprises blood or a blood fraction.

5. The method according to claim 4, wherein the blood fraction comprises purified peripheral blood mononuclear cells (PBMCs).

6. The method according to claim 5, wherein the PBMCs are CD14+/CD16−, CD14−/CD16+, or CD14+/CD16+.

7. The method according to claim 1 further comprising:
selecting an individual prior to the individual receiving an anti-TNFα therapy and prior to said obtaining.

8. The method according to claim 1 further comprising:
selecting an individual showing an inadequate response to a non-TNFα inhibiting therapeutic agent after at least 4 months of therapy prior to said obtaining.

9. The method according to claim 1 further comprising:
selecting an individual exhibiting one or more clinical symptoms of arthritis or ankylosing spondylitis prior to said obtaining.

10. The method according to claim 9, wherein the one or more clinical symptoms comprise synovitis in at least one joint, seropositivity for rheumatoid factor, seropositivity for anti-citrullinated protein antibody, abnormal C-reactive protein, abnormal erythrocyte sedimentation rate, current psoriasis or personal or family history of psoriasis, psoriatic nail dystrophy, negative rheumatoid factor, dactylitis, low back pain and stiffness, pain and stiffness in the thoracic region, limited motion in the lumbar spine, limited chest expansion, and history or evidence of iritis or uveitis.

11. The method according to claim 1 further comprising:
contacting the sample obtained from the individual with one or more reagents capable of binding TRAF3 in the sample prior to said determining.

12. The method according to claim 11, wherein at least one of the one or more reagents is coupled to a detectable label.

13. The method according to claim 11, wherein the one or more reagents comprise a primary TRAF3 binding reagent.

14. The method according to claim 13, wherein the primary TRAF3 binding reagent is coupled to a detectable label by a secondary binding reagent having binding specificity for the primary binding reagent.

15. The method according to claim 13, wherein the primary TRAF3 binding reagent comprises an anti-TRAF3 antibody, binding fragment thereof, or polypeptide or non-polypeptide antibody mimic.

16. The method according to claim 11 wherein said determining comprises:
measuring the expression level of TRAF3 in the sample based on said contacting.

17. The method according to claim 1 further comprising:
comparing the expression of TRAF3 in the biological sample to a control level of TRAF3.

18. The method according to claim 17, wherein the control level is the average level or normal range of TRAF3 expression in healthy individuals.

19. A method of identifying an individual that has an immune disorder and is likely to respond to anti-TNFα therapy, said method comprising:
obtaining a biological sample from the individual;
labeling TRAF3 in the sample with a TRAF3 binding reagent; and
determining a level of TRAF3 in the sample based on said labeling, wherein an elevated TRAF3 level identifies the individual as likely to respond to anti-TNFα therapy.

20. The method according to claim 19, wherein the immune disorder is selected from the group of inflammatory bowel diseases, rheumatoid arthritis, psoriatic arthritis, and ankylosing spondylitis.

21. The method according to claim 20, wherein the immune disorder is rheumatoid arthritis (RA) or psoriatic arthritis (PsA).

22. The method according to claim 19, wherein the biological sample comprises blood or a blood fraction.

23. The method according to claim 22, wherein the blood fraction comprises peripheral blood mononuclear cells (PBMCs).

24. The method according to claim 23, wherein the PBMCs are CD14+/CD16−, CD14−/CD16+, or CD14+/CD16+.

25. The method according to claim 19, wherein the TRAF3 binding reagent is selected from the group of antibody, binding fragment thereof, or polypeptide or non-polypeptide antibody mimic.

26. The method according to claim 19 further comprising:
selecting an individual prior to the individual receiving an anti-TNFα therapy and prior to said obtaining.

27. The method according to claim 19 further comprising:
selecting an individual showing an inadequate response to a non-TNFα inhibiting therapeutic agent after at least 4 months of therapy prior to said obtaining.

28. The method according to claim 19 further comprising:
selecting an individual exhibiting one or more clinical symptoms of arthritis or ankylosing spondylitis prior to said obtaining.

29. The method according to claim 28, wherein the one or more clinical symptoms comprise synovitis in at least one joint, seropositivity for rheumatoid factor, seropositivity for anti-citrullinated protein antibody, abnormal C-reactive protein, abnormal erythrocyte sedimentation rate, current psoriasis or personal or family history of psoriasis, psoriatic nail dystrophy, negative rheumatoid factor, dactylitis, low back pain and stiffness, pain and stiffness in the thoracic region, limited motion in the lumbar spine, limited chest expansion, and history or evidence of iritis or uveitis.

30. The method according to claim 19, wherein the TRAF3 binding reagent is coupled to a detectable label.

31. The method according to claim 30, wherein the TRAF3 binding reagent is coupled to a detectable label by a secondary binding reagent having binding specificity for the TRAF3 binding reagent.

32. The method according to claim 19 further comprising:
measuring the expression level of TRAF3 in the biological sample based on said labeling.

33. The method according to claim 32 further comprising:
comparing the expression of TRAF3 in the biological sample to a control level of TRAF3.

34. The method according to claim 33, wherein the control level is the average level or normal range of TRAF3 expression in healthy individuals.

35. A method of treating a patient for an immune disorder, said method comprising:
determining a level of TRAF3 in a patient sample; and
administering to the patient a suitable therapeutic regimen to treat said immune disorder, wherein the suitable therapeutic regiment is selected for said administering based on the TRAF3 level in the patient sample.

36. The method according to claim 35, wherein the immune disorder is selected from the group consisting of inflammatory bowel diseases, rheumatoid arthritis, psoriatic arthritis, and ankylosing spondylitis.

37. The method according to claim 36, wherein the immune disorder is rheumatoid arthritis (RA) or psoriatic arthritis (PsA).

38. The method according to claim 35, wherein the patient sample comprises blood or a blood fraction.

39. The method according to claim 38, wherein the blood fraction comprises peripheral blood mononuclear cells (PBMCs).

40. The method according to claim 39, wherein the PBMCs are CD14+/CD16−, CD14−/CD16+, or CD14+/CD16+.

41. The method according to claim 35, wherein, when the patient is determined to have an elevated level of TRAF3, said therapeutic regimen comprises an anti-TNFα therapeutic agent.

42. The method according to claim 41, wherein the anti-TNFα therapeutic agent is selected from the group of infliximab, etanercept, adalimumab, certolizumab, or golimumab.

43. The method according to claim 42, wherein the therapeutic regimen further comprises a non-steroidal anti-inflammatory therapeutic agent or a corticosteroid.

44. The method according to claim 35, wherein, when the patient is determined to have a low level of TRAF3, said therapeutic regimen comprises an anti B-cell, anti T-cell, anti IL-1, anti IL-6, or anti-malarial therapeutic agent.

45. The method according to claim 44, wherein the therapeutic regimen is selected from the group of methotrexate, chloroquine, hydroxychloroquine, sulfasalazine, minocycline, azathioprine, cyclosporine, cyclophosphamide, anakinra, abatacept, rituximab, or tocilizumab.

46. The method according to claim 45, wherein the therapeutic regimen further comprises a non-steroidal anti-inflammatory therapeutic agent or a corticosteroid.

47. The method according to claim 35 further comprising:
contacting the patient sample with one or more reagents capable of binding TRAF3 in the sample prior to said determining.

48. The method according to claim 47, wherein at least one of the one or more reagents is coupled to a detectable label.

49. The method according to claim 47, wherein the one or more reagents comprise a primary TRAF3 binding reagent.

50. The method according to claim 49, wherein the primary TRAF3 binding reagent is coupled to a detectable label by a secondary reagent having binding specificity for the primary TRAF3 binding reagent.

51. The method according to claim 49, wherein the primary TRAF3 binding reagent comprises an anti-TRAF3 antibody, binding fragment thereof, or polypeptide or non-polypeptide antibody mimic.

52. The method according to claim 47 further comprising:
measuring the expression level of TRAF3 in the patient sample based on said contacting.

53. The method according to claim 52 further comprising:
comparing the expression of TRAF3 in the patient sample to a control level of TRAF3.

54. The method according to claim 53, wherein the control level is the average level or normal range of TRAF3 expression in healthy individuals.
